(19) United States
(12) Patent Application Publication
(10) Pub. No.: US 2024/0000323 A1
(43) Pub. Date: Jan. 4, 2024

(54) PHOTOPLETHYSMOGRAPHY-BASED REAL-TIME BLOOD PRESSURE MONITORING SYSTEM USING CONVOLUTIONAL BIDIRECTIONAL SHORT- AND LONG-TERM MEMORY RECURRENT NEURAL NETWORK, AND REAL-TIME BLOOD PRESSURE MONITORING METHOD USING SAME

(71) Applicant: Korea University Research and Business Foundation, Seoul (KR)

(72) Inventors: Dong-Joo KIM, Seoul (KR); Dong-Kyu KIM, Seoul (KR); Young Tak KIM, Seoul (KR); Se Ho LEE, Seoul (KR)

(73) Assignee: Korea University Research and Business Foundation, Seoul (KR)

(21) Appl. No.: 18/031,286

(22) PCT Filed: Sep. 29, 2021
(86) PCT No.: PCT/KR2021/013286

§ 371 Date: Apr. 11, 2023

(30) Foreign Application Priority Data
Oct. 22, 2020 (KR) 10-2020-0137715
Nov. 30, 2020 (KR) 10-2020-0164778

(51) Int. Cl. A61B 5/021; G16H 50/20

(52) U.S. Cl. CPC A61B 5/02108; G16H 50/20

(57) ABSTRACT

The present disclosure relates to a real-time blood pressure monitoring system based on photoplethysmography (PPG) using convolutional⋅bidirectional long short-term memory (LSTM) recurrent neural networks and a real-time blood pressure monitoring method using the same. According to the present disclosure, there is provided the real-time blood pressure monitoring system based on PPG using convolutional⋅bidirectional LSTM recurrent neural networks, including a pulse wave measurement module configured to measure the PPG, and a blood pressure estimation server configured to receive the measured PPG from the pulse wave measurement module and estimate a blood pressure via the recurrent neural networks.

Additionally, there is provided the real-time blood pressure monitoring method using the real-time blood pressure monitoring system based on PPG using convolutional⋅bidirectional LSTM recurrent neural networks, the method including a measurement step of measuring the PPG through the pulse wave measurement module, and an estimation step of estimating, by the blood pressure estimation server, a blood pressure through the recurrent neural networks using the PPG.

### TECHNICAL FIELD

The present disclosure relates to a real-time blood pressure monitoring system based on photoplethysmography (PPG) using convolutional⋅bidirectional long short-term memory (LSTM) recurrent neural networks and a real-time blood pressure monitoring method using the same, and more particularly, to a real-time blood pressure monitoring system based on PPG using convolutional⋅bidirectional LSTM recurrent neural networks for estimation and monitoring of invasive arterial blood pressure in real time based on PPG which is a non-invasive measurement method, thereby estimating the whole blood pressure including systolic diastolic blood pressure and achieving continuous monitoring, and a real-time blood pressure monitoring method using the same.

### BACKGROUND ART

The article “Blood Pressure Estimation Using Photoplethysmogram Signal and Its Morphological Features” from IEEE Sensors Journal in 2019 describes a method for estimating the systolic diastolic blood pressure through photoplethysmography feature information learning. The corresponding article presents calculation of feature information such as slope, interval, amplitude, etc. from non-invasive photoplethysmography (PPG) and estimation of systolic diastolic blood pressure through machine learning.

Additionally, the article “The use of photoplethysmography for assessing hypertension” from NPJ digital medicine in 2019 describes a method for estimating the systolic blood pressure through two signals, i.e., PPG and electrocardiography (ECG) signals. In the corresponding article, the systolic blood pressure is estimated by making use of PPG and ECG signals that are easier than invasive blood pressure measurement and simultaneous learning through deep learning.

However, since the first technology needs the process of calculating and processing the feature information, and the second technology requires a large amount of computational resources due to the use of two signals, the two technologies lack real time performance. Additionally, since only highest and lowest blood pressure within a set interval is estimated, continuous blood pressure monitoring is impossible.

Accordingly, there is a need for development of technology to continuously monitor the blood pressure by estimating the whole blood pressure in real time.

### DISCLOSURE

### Technical Problem

To solve the above-described problem, the present disclosure is directed to providing a real-time blood pressure monitoring system based on photoplethysmography (PPG) using convolutional⋅bidirectional long short-term memory (LSTM) recurrent neural networks for estimation and monitoring of invasive arterial blood pressure in real time based on PPG which is a non-invasive measurement method, thereby estimating the whole blood pressure including systolic⋅diastolic blood pressure and achieving continuous monitoring, and a real-time blood pressure monitoring method using the same.

### Technical Solution

To solve the above-described problem, there is provided a real-time blood pressure monitoring system based on photoplethysmography (PPG) using convolutional⋅bidirectional long short-term memory (LSTM) recurrent neural networks according to an embodiment of the present disclosure, including a pulse wave measurement module configured to measure the PPG, and a blood pressure estimation server configured to receive the measured PPG from the pulse wave measurement module and estimate a blood pressure via the recurrent neural networks.

Here, the pulse wave measurement module may measure the PPG using a near-infrared sensor.

Additionally, the recurrent neural networks may be trained with big data which is a collection of the blood pressure measured through A-line and the PPG measured for a same time period.

Additionally, the recurrent neural networks may estimate the blood pressure according to the input PPG by a many to many architecture of convolutional neural network (CNN) and bidirectional LSTM recurrent neural network.

Additionally, the recurrent neural networks may include at least one CNN to extract multidimensional information from the input PPG, and at least one bidirectional LSTM recurrent neural network to estimate the blood pressure through the extracted multidimensional information.

Additionally, the real-time blood pressure monitoring system may further include a monitoring terminal configured to receive the estimated blood pressure from the blood pressure estimation server.

In addition, there is provided a real-time blood pressure monitoring method using a real-time blood pressure monitoring system based on PPG using convolutional⋅bidirectional LSTM recurrent neural networks according to an embodiment of the present disclosure, the method including a measurement step of measuring the PPG through a pulse wave measurement module, and an estimation step of estimating, by a blood pressure estimation server, a blood pressure through the recurrent neural networks using the PPG.

Additionally, the real-time blood pressure monitoring method may further include, after the estimation step, a monitoring step of monitoring, by a monitoring terminal, the blood pressure by receiving the estimated blood pressure from the blood pressure estimation server.

Additionally, the estimation step may include an information extraction step of extracting, by the blood pressure estimation server, multidimensional information from the input PPG via at least one CNN, and a blood pressure estimation step of estimating, by the blood pressure estimation server, the blood pressure from the extracted multidimensional information via at least one bidirectional LSTM recurrent neural network.

Additionally, the real-time blood pressure monitoring method may further include, after the estimation step, an analysis step of analyzing, by the blood pressure estimation server, the estimated blood pressure to generate blood pressure analysis information.

### Advantageous Effects

The real-time blood pressure monitoring system based on photoplethysmography (PPG) using convolutional⋅bidirectional long short-term memory (LSTM) recurrent neural networks according to an embodiment of the present disclosure and the real-time blood pressure monitoring method using the same performs estimation of invasive arterial blood pressure based on PPG which is a non-invasive measure method, thereby achieving real-time estimation with a small amount of computational resources using only the raw PPG signal without any calculation.

Additionally, it is possible to estimate the whole blood pressure including systolic/diastolic blood pressure and achieve continuous monitoring. Accordingly, it may be used as an index for identifying risks of cardiovascular diseases.

Additionally, since it is possible to estimate the blood pressure by a non-invasive method, it can be easily used in daily life, so patients having high incidence of diseases can monitor the blood pressure in real time and control the blood pressure.

### DESCRIPTION OF DRAWINGS

FIG. 1 is an architecture diagram showing a real-time blood pressure monitoring system based on photoplethysmography (PPG) using convolutional⋅bidirectional long short-term memory (LSTM) recurrent neural networks according to an embodiment of the present disclosure.

FIG. 2 is a block diagram showing a blood pressure estimation server of FIG. 1.

FIGS. 3A and 3B are diagrams showing data of PPG and arterial blood pressure (ABP) measured per person for the same time period to collect big data.

FIG. 4 is a diagram showing convolutional⋅bidirectional LSTM recurrent neural networks according to an embodiment of the present disclosure.

FIG. 5 is a flowchart schematically showing a real-time blood pressure monitoring method using a real-time blood pressure monitoring system based on PPG using convolutional⋅bidirectional LSTM recurrent neural networks according to an embodiment of the present disclosure.

FIGS. 6A and 6B are error graphs showing the comparison between actual blood pressure and estimated systolic blood pressure (SBP) and diastolic blood pressure (DBP) through a real-time blood pressure monitoring system based on PPG using convolutional⋅bidirectional LSTM recurrent neural networks according to an embodiment of the present disclosure.

FIGS. 7A to 7C are graphs showing the comparison between actually measured arterial blood pressure and estimated ABP through a real-time blood pressure monitoring system based on PPG using convolutional⋅bidirectional LSTM recurrent neural networks according to an embodiment of the present disclosure.

### BEST MODE

Hereinafter, the present disclosure described with reference to the accompanying drawings is not limited to a particular embodiment, and a variety of modifications may be made thereto, so the present disclosure may have a plurality of embodiments. Additionally, it should be understood that the following description includes all modifications, equivalents or substitutions included in the spirit and scope of the present disclosure.

In the following description, the term such as first, second or the like is used to describe a variety of elements, its meaning is not limited by the term itself, and the term is used to distinguish one element from another.

Like reference numbers used throughout the specification indicate like elements.

Unless the context clearly indicates otherwise, the singular form as used herein includes the plural form. Additionally, the term “comprising”, “including” or “having” when used in this specification, should be interpreted as specifying the presence of stated features, integers, steps, operations, elements, components or a combination thereof, and it should be understood that the term does not preclude the presence or addition of one or more other features, integers, steps, operations, elements, components or a combination thereof.

Additionally, the term “unit”, “-er/or”, “module” as used herein refers to a processing unit of at least one function or operation, and this may be incorporated in hardware, software or a combination of hardware and software.

Hereinafter, a real-time blood pressure monitoring system based on photoplethysmography (PPG) using convolutional⋅bidirectional long short-term memory (LSTM) recurrent neural networks according to an embodiment of the present disclosure and a real-time blood pressure monitoring method using the same will be described in detail with reference to the accompanying drawings.

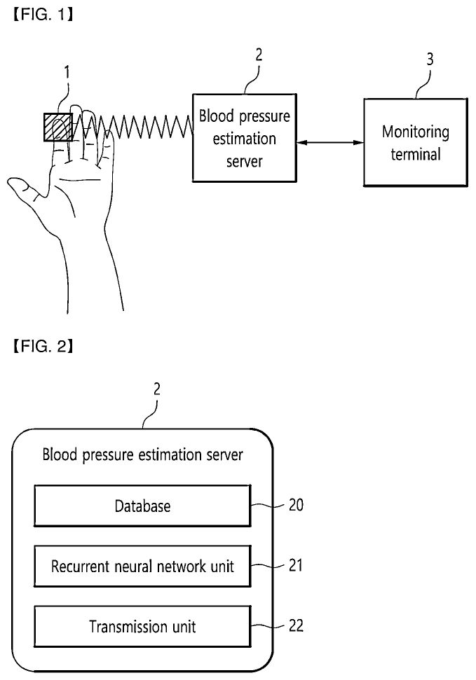

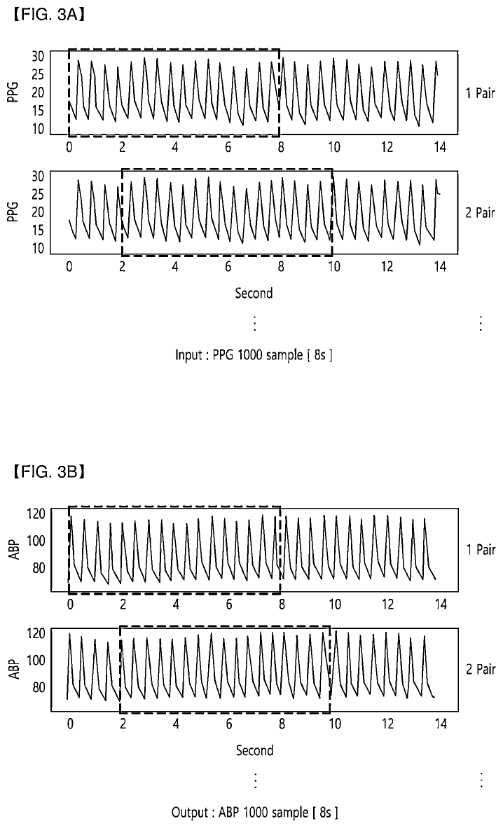

FIG. 1 is an architecture diagram showing a real-time blood pressure monitoring system based on PPG using convolutional⋅bidirectional LSTM recurrent neural networks according to an embodiment of the present disclosure, FIG. 2 is a block diagram showing a blood pressure estimation server of FIG. 1, FIGS. 3A and 3B are diagrams showing data of PPG and arterial blood pressure (ABP) measured per person for the same time period to collect big data, and FIG. 4 is a diagram showing convolutional⋅bidirectional LSTM recurrent neural networks according to an embodiment of the present disclosure.

Referring to FIG. 1, the real-time blood pressure monitoring system based on PPG using convolutional⋅bidirectional LSTM recurrent neural networks according to an embodiment of the present disclosure may include a pulse wave measurement module 1, a blood pressure estimation server 2 and a monitoring terminal 3.

The pulse wave measurement module 1 may measure PPG using a near-infrared sensor.

Here, the PPG may be measured by measuring blood volume changes using near-infra red light by a non-invasive method, and since the PPG measures the quantity of blood flowing according to the heart beats and the elasticity of the blood vessel, it is primarily used to monitor heart rate variability.

Additionally, the pulse wave measurement module 1 may transmit the measured PPG to the blood pressure estimation server 2. It allows the blood pressure estimation server 2 to estimate the blood pressure using the PPG.

The blood pressure estimation server 2 may receive the measured PPG from the pulse wave measurement module 1 and estimate the blood pressure via a recurrent neural network. The used recurrent neural network is convolutional⋅bidirectional LSTM recurrent neural networks and will be described in more detail below.

Referring to FIG. 2, the blood pressure estimation server 2 may include a database 20, a recurrent neural network unit 21 and a transmission unit 22.

As shown in FIG. 3, the database 20 may store big data collected by measuring the blood pressure (measured through A-line) and PPG per person for the same time period. When measuring the PPG and the blood pressure per person, data may be collected at 125 Hz, but is not limited thereto. The blood pressure is preferably ABP, but is not limited thereto.

The recurrent neural network is trained with the big data to estimate the blood pressure, and may be trained with data of each of the PPG and ABP included in data of a long time period at an interval of a few seconds, and the time interval is preferably 8 seconds, but is not limited thereto.

Additionally, the database 20 may store all information necessary for the system, such as blood pressure reference information. Here, the blood pressure reference information may include normal blood pressure values by at least one of disease, age or gender.

The recurrent neural network unit 21 may estimate the blood pressure from the PPG via the recurrent neural network.

Here, the recurrent neural network is convolutional⋅bidirectional LSTM recurrent neural networks, and may include the convolutional neural network (CNN) and the bidirectional LSTM recurrent neural network in a many to many architecture.

The CNN is a deep learning model used to extract multidimensional information from data, and may be used to extract multiple pieces of information from one-dimensional PPG signal data.

More specifically, the PPG is one-dimensional time-series data that is a sequence of values over time, and two-dimensional temporal information may be extracted from the one-dimensional data via the CNN.

The CNN has a filter, and feature maps may be generated to extract features by moving the filter at a regular interval, and multidimensional information may be extracted in two dimensions from one dimension through the trained feature maps. Here, since the plurality of feature maps share their training weights, it is possible to learn the overall phase and shape of the PPG, thereby extracting the overall phase and shape of the PPG using the multidimensional information.

The LSTM recurrent neural network is a model that passes the memory state as input to the next neural network to prevent the previous information loss when learning long data in a sequential order, and is primarily used in time-series data. Since bidirectional learning includes forward learning and backward learning, it is possible to achieve more diverse information learning in signal data of time series.

As described above, the CNN extracts the overall phase and shape information of the PPG, and the LSTM is a model that learns the previous and subsequent parts of a particular location, and thus it is characterized by learning the extracted information via the CNN in a sequential order, and learning using bidirectional information ( . . . , t−3, t−2, t−1, . . . , t+1, t+2, t+3, . . . ) together when learning the specific time t. The corresponding number of estimated values to the number of data of ABP collected as big data are produced through learning via at least one LSTM, and the learning may be performed to minimize errors between the estimated and actual values.

Meanwhile, the many to many architecture of the recurrent neural network is a deep learning technique that learns multiple inputs in a sequential order and yields multiple outputs.

As described above, the present disclosure builds the recurrent neural network in the many to many architecture using the two models to accept the input PPG and yield the estimated blood pressure output, thereby estimating the blood pressure with a small amount of computational resources, and achieving real-time estimation and whole estimation.

More specifically, the recurrent neural network includes at least one CNN and at least one bidirectional LSTM recurrent neural network connected in that order, and may extract multidimensional information from the input PPG via the at least one CNN, and estimate the blood pressure from the extracted multidimensional information via the at least one bidirectional LSTM recurrent neural network.

As shown in FIG. 4, each of the CNN and the bidirectional LSTM recurrent neural network includes two layers, and the ABP is preferably estimated through a dense layer, but is not limited thereto.

The transmission unit 22 may transmit the estimated blood pressure to the monitoring terminal 3 through the recurrent neural network unit 21. In this instance, the estimated blood pressure value may be transmitted in text, but may be transmitted in the form of a variety of graphs and tables. Additionally, the transmission unit 22 may transmit blood pressure analysis information to the monitoring terminal 3.

Additionally, the blood pressure estimation server 2 may further include an analysis unit (not shown).

The analysis unit may analyze the estimated blood pressure based on the blood pressure reference information to generate the blood pressure analysis information. This may allow a user to determine his/her blood pressure condition and how to control the blood pressure.

For example, the blood pressure analysis information may include risks of cardiovascular diseases and desirable blood pressure values, but is not limited thereto, and may further include various pieces of information such as foods/activities to avoid, necessary foods/activities, etc.

The monitoring terminal 3 receives the estimated blood pressure from the blood pressure estimation server 2 and outputs it to allow the user to see the estimated blood pressure. Additionally, the monitoring terminal 3 may receive the blood pressure analysis information from the blood pressure estimation server 2.

A method for monitoring the blood pressure in real time using the real-time blood pressure monitoring system based on PPG using convolutional⋅bidirectional LSTM recurrent neural networks will be described in detail below.

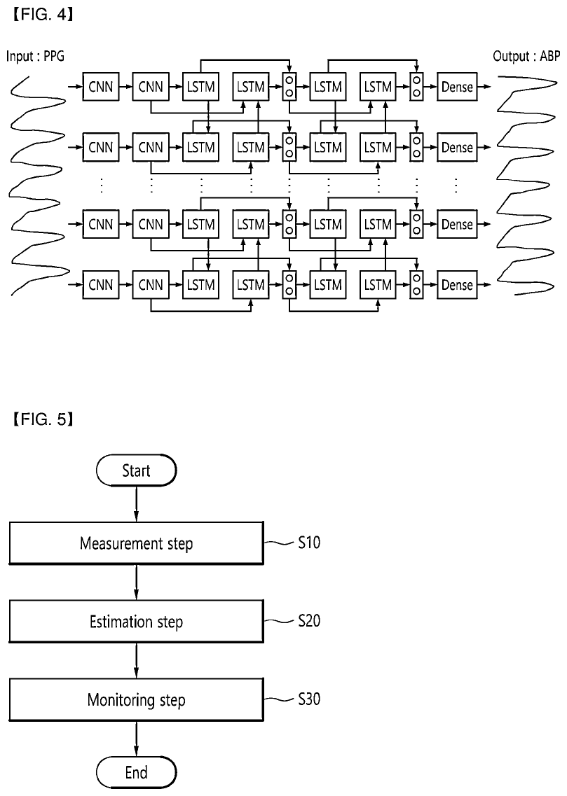

FIG. 5 is a flowchart schematically showing the real-time blood pressure monitoring method using the real-time blood pressure monitoring system based on PPG using convolutional⋅bidirectional LSTM recurrent neural networks according to an embodiment of the present disclosure.

Referring to FIG. 5, the real-time blood pressure monitoring method using the real-time blood pressure monitoring system based on PPG using convolutional⋅bidirectional LSTM recurrent neural networks according to an embodiment of the present disclosure may include a measurement step (S10), an estimation step (S20) and a monitoring step (S30).

To begin with, the measurement step (S10) may include measuring the user's PPG through the pulse wave measurement module 1. The measured PPG may be transmitted to the blood pressure estimation server 2.

The estimation step (S20) may include estimating, by the blood pressure estimation server 2, the blood pressure via the recurrent neural network using the received PPG. The recurrent neural network has been described above in detail, and its detailed description is omitted.

The step S20 may include an information extraction step and a blood pressure estimation step.

The information extraction step may include extracting, by the blood pressure estimation server 2, multidimensional information from the input PPG via the at least one CNN of the recurrent neural network.

The blood pressure estimation step may include estimating, by the blood pressure estimation server, the blood pressure from the extracted multidimensional information via the at least one bidirectional LSTM recurrent neural network of the recurrent neural network.

The estimated blood pressure may be transmitted to the monitoring terminal 3.

The monitoring step (S30) may include receiving, by the monitoring terminal 3, the estimated blood pressure from the blood pressure estimation server to allow the user or measurer to monitor.

Additionally, the real-time blood pressure monitoring method according to an embodiment of the present disclosure may further include an analysis step (not shown) after the step S20.

The analysis step may include analyzing, by the blood pressure estimation server 2, the estimated blood pressure to generate blood pressure analysis information. The generated blood pressure analysis information may be transmitted to the monitoring terminal 3.

As described above, the real-time blood pressure monitoring system based on PPG using convolutional⋅bidirectional LSTM recurrent neural networks according to an embodiment of the present disclosure and the real-time blood pressure monitoring method using the same may achieve real-time estimation with a small amount of computational resources using only the raw PPG signal without any calculation by the estimation of invasive arterial blood pressure based on PPG which is a non-invasive measurement method.

Additionally, it is possible to estimate the whole blood pressure including systolic/diastolic blood pressure and achieve continuous monitoring. Accordingly, it may be used as an index for identifying risks of cardiovascular diseases.

Additionally, since it is possible to estimate the blood pressure by a non-invasive method, it can be easily used in daily life, so patients having high incidence of diseases can monitor the blood pressure in real time and control the blood pressure.

Hereinafter, the present disclosure described above will be described in more detail through experimental examples and examples. However, the present disclosure is not necessarily limited to these experimental examples and examples.

### [Experimental Example 1] Error Evaluation of Systolic Blood Pressure and Diastolic Blood Pressure

To evaluate the performance of an example of the present disclosure, in the example of the present disclosure, Systolic Blood Pressure (SBP) and Diastolic Blood Pressure (DBP) are estimated, and a Mean Absolute Error (MAE) is calculated by comparing the estimated SBP and DBP with the actually measured SBP and DBP and then compared.

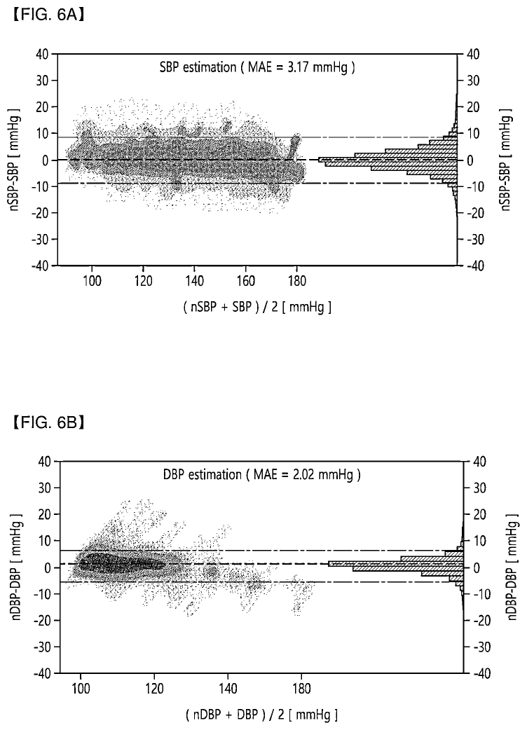

The results are shown in Table 1 and FIGS. 6A and 6B.

TABLE 1

| | Mean Absolute Eroor[MAE] | | |
|---|---|---|---|
| | SBP | DBP | MAP |
| [symbol] | 3.17 | 2.02 | 1.32 |

As can be seen from the above Table 1, in the example, the error in SBP and DBP is less than 5, and a very low error of 1.32 is found in the evaluation index, Mean Arterial Pressure (MAP), showing the outstanding performance.

FIGS. 6A and 6B are error statistics graphs of the actual blood pressure and the estimated SBP and DBP through the real-time blood pressure monitoring system based on PPG using convolutional⋅bidirectional LSTM recurrent neural networks according to an embodiment of the present disclosure, and it can be seen that both the estimated SBP and DBP is close to 0. This confirms that the system of the present disclosure has high accuracy of blood pressure estimation.

### [Experimental Example 2] Comparison of Estimated Blood Pressure and Actual Blood Pressure

To evaluate the performance of an example of the present disclosure, after estimating the ABP of three subjects through the real-time blood pressure monitoring system of the present disclosure and actually measuring the ABP, the estimated blood pressure and the actual blood pressure are compared.

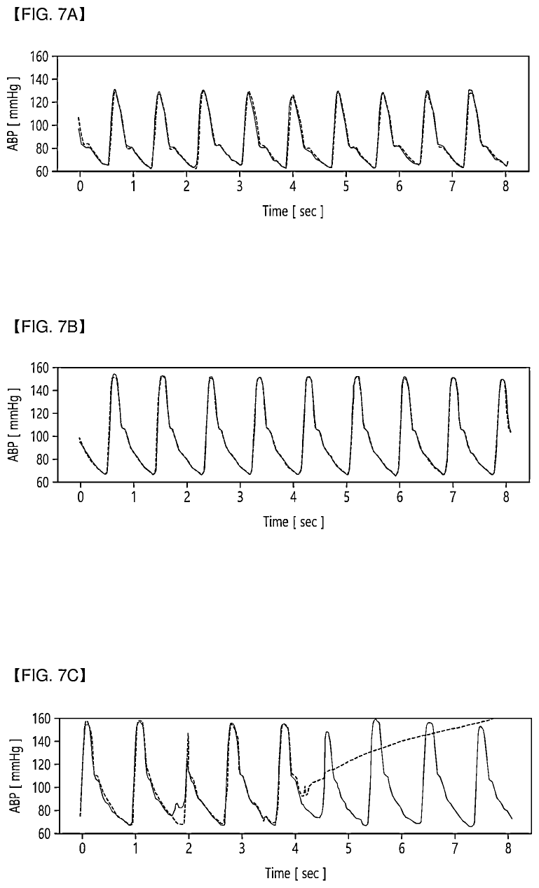

The results are shown in FIGS. 7A to 7C.

As can be seen from FIGS. 7A to 7C, the shape and phase in the graph of the estimated blood pressure are almost identical to the shape and phase in the graph of the actual blood pressure (FIGS. 7A and 7B).

Additionally, seeing FIG. 7C, it can be seen that it is possible to estimate the ABP through PPG even in the event of artifacts (outlier values) during invasive blood pressure measurement. The artifacts may occur when the subject makes a motion or a catheter is replaced.

That is, it is confirmed that the real-time blood pressure monitoring system of the present disclosure estimates the blood pressure almost equally to the actual blood pressure.

While the embodiments of the present disclosure have been hereinabove with reference to the accompanying drawings, those skilled in the art will understand that the present disclosure may be embodied in other particular forms without changing the technical spirit or essential feature of the present disclosure. Accordingly, the above-described embodiments are provided by way of illustration, and not limitation, in all aspects.

### DETAILED DESCRIPTION OF MAIN ELEMENTS

1: Pulse wave measurement module
2: Blood pressure estimation server
20: Database
21: Recurrent neural network unit
22: Transmission unit
3: Monitoring terminal

1. A real-time blood pressure monitoring system based on photoplethysmography (PPG) using convolutional⋅bidirectional long short-term memory (LSTM) recurrent neural networks, comprising:
a pulse wave measurement module configured to measure the PPG; and
a blood pressure estimation server configured to receive the measured PPG from the pulse wave measurement module and estimate a blood pressure via the recurrent neural networks.

2. The real-time blood pressure monitoring system according to claim 1, wherein the pulse wave measurement module measures the PPG using a near-infrared sensor.

3. The real-time blood pressure monitoring system according to claim 1, wherein the recurrent neural networks are trained with big data which is a collection of the blood pressure measured through A-line and the PPG measured for a same time period.

4. The real-time blood pressure monitoring system according to claim 1, wherein the recurrent neural networks estimate the blood pressure according to the input PPG by a many to many architecture of convolutional neural network (CNN) and bidirectional LSTM recurrent neural network.

5. The real-time blood pressure monitoring system according to claim 1, wherein the recurrent neural networks include:
at least one CNN to extract multidimensional information from the input PPG; and
at least one bidirectional LSTM recurrent neural network to estimate the blood pressure through the extracted multidimensional information.

6. The real-time blood pressure monitoring system according to claim 1, further comprising:
a monitoring terminal configured to receive the estimated blood pressure from the blood pressure estimation server.

7. A real-time blood pressure monitoring method using a real-time blood pressure monitoring system based on photoplethysmography (PPG) using convolutional⋅bidirectional long short-term memory (LSTM) recurrent neural networks, the method comprising:
a measurement step of measuring the PPG through a pulse wave measurement module; and
an estimation step of estimating, by a blood pressure estimation server, a blood pressure through the recurrent neural networks using the PPG.

8. The real-time blood pressure monitoring method according to claim 7, after the estimation step, further comprising:
a monitoring step of monitoring, by a monitoring terminal, the blood pressure by receiving the estimated blood pressure from the blood pressure estimation server.

9. The real-time blood pressure monitoring method according to claim 7, wherein the estimation step comprises:
an information extraction step of extracting, by the blood pressure estimation server, multidimensional information from the input PPG via at least one convolutional neural network (CNN); and
a blood pressure estimation step of estimating, by the blood pressure estimation server, the blood pressure from the extracted multidimensional information via at least one bidirectional LSTM recurrent neural network.

10. The real-time blood pressure monitoring method according to claim 7, after the estimation step, further comprising:
an analysis step of analyzing, by the blood pressure estimation server, the estimated blood pressure to generate blood pressure analysis information.
